(19) United States
(12) Patent Application Publication
(10) Pub. No.: US 2022/0079982 A1
(43) Pub. Date: Mar. 17, 2022

(54) FORMULATION FOR TREATING TOBACCO OR PSYCHOTROPE WITHDRAWAL SYMPTOMS

(71) Applicant: EDELWEISS LABORATORY SA, SION (FR)

(72) Inventor: ROBERT ABOUDARAM, BOULOGNE-BILLANCOURT (FR)

(21) Appl. No.: 17/423,086

(22) PCT Filed: Jan. 17, 2020
(86) PCT No.: PCT/EP2020/051168

§ 371 Date: Jul. 14, 2021

(30) Foreign Application Priority Data
Jan. 17, 2019 (FR) 1900425

(51) Int. Cl. A61K 33/242; A61K 36/34; A61K 36/53; A61K 36/56; A61K 36/81

(52) U.S. Cl. CPC A61K 33/242; A61K 36/34; A61K 36/81; A61K 36/56; A61K 36/53

(57) ABSTRACT

The invention relates to a homoeopathic formulation prepared from the six homoeopathic strains tabacum, Nux vomica, Aurum metallicum, Passiflora incarnata, Agnus castus and Lobelia inflata, said homoeopathic formulation having beneficial effects on persons undergoing smoking or psychotropic drug withdrawal.

### TECHNICAL FIELD OF THE INVENTION

The invention relates to the field of medicines used for the treatment of symptoms related to smoking cessation and/or psychotropic drug withdrawal, and of medicines used to accompany people wishing to stop or reduce smoking and/or psychotropic drug consumption.

In particular, the invention relates to a homoeopathic medicine comprising a mixture obtained from 6 homoeopathic strains.

The use of the homoeopathic medicine in the treatment of symptoms related to smoking cessation and/or psychotropic drug withdrawal, and its use in accompanying weaned persons, are also subjects of the present invention.

Different pharmaceutical forms, different routes of administration and different medicine combinations can be envisaged in the context of the present invention.

### PRIOR ART

Smoking and substance abuse are preventable causes of serious diseases such as cancer, cardiovascular accidents, respiratory disorders, strokes and overdoses. Despite major prevention efforts to warn of the dangers of such consumption in some countries, notably through information campaigns, smoking, for example, has only moderately declined.

The use of tobacco and certain psychotropic substances is highly addictive, and this from the very first time of use. The strong addiction caused by the consumption of these products makes it difficult to stop using them. For example, in the case of tobacco users, and despite a declared desire to stop consumption, or to at least reduce consumption, it is not uncommon for the addicted person to fail to reduce or stop consumption, or to return to consumption after a more or less extensive period of withdrawal.

In addition to various behavioural therapies, or alternative medicines such as acupuncture, three main types of treatments currently exist to treat tobacco addiction:

Consumption of nicotine substitutes, for example via dermal patches diffusing these derivatives through the epidermis, or by chewing gum;

Buproprion, notably sold as a medicine under the trade name Zyban®;

Homeopathy.

For each of these methods, the mechanism of action to reduce or even eliminate the effects of smoking cessation consists of the use of nicotine molecules or nicotine analogues. Nicotine is a substance found in cigarettes and is involved in smoking addiction. Present on the surface of certain neurons, nicotinic acetylcholine receptors are activated when they bind nicotine; for example when a smoker consumes a cigarette. The activation of these receptors results in the release of dopamine, a hormone that imparts a feeling of satisfaction. Thus, the brain associates smoking with immediate satisfaction.

Thus, when nicotine is no longer sufficiently present in the body, the latter emits biological signals that make it feel the need for this substance, leading to the appearance of an urgent and imperative need to smoke a cigarette.

When a smoker stops smoking, many symptoms appear because of this withdrawal syndrome: mood disorders, particularly a state of depression or sadness, anxiety, nervousness, irritability, frustration, anger, impatience, restlessness, mood swings, insomnia, difficulty concentrating, an increase in appetite often accompanied by a significant weight gain.

Although there are some medicines available to help a person stop or reduce smoking, there is still a need for more effective medicines, for example to reduce the number of relapses, or to reduce the intensity of withdrawal symptoms, or to reduce the duration of these symptoms. The present invention is therefore part of the search for new treatments to accompany smoking cessation.

### DISCLOSURE OF THE INVENTION

The invention is therefore intended to address, at least in part, the shortcomings of the prior art.

In particular, the invention is intended to provide a formulation that reduces at least some of the symptoms that occur when a person is weaned off tobacco. Another purpose of the invention is to provide a formulation that reduces the intensity of at least some of the symptoms that occur with smoking cessation. Another purpose of the invention is to provide a formulation for reducing the duration of at least some of the symptoms caused by smoking cessation.

### SUMMARY OF THE INVENTION

The invention is defined by the independent claims. The dependent claims define preferred embodiments of the invention.

According to a first aspect, the invention concerns a homoeopathic medicinal formulation prepared from 6 homoeopathic strains.

The homoeopathic composition according to this first aspect of the invention comprises, in equal masses:

One mother tincture of tabacum, optionally diluted up to a factor of 60C;

One mother tincture of Nux vomica, optionally diluted up to a factor of 60C;

One mother tincture of Aurum metallicum, optionally diluted up to a factor of 60C;

One mother tincture of Passiflora incarnata, optionally diluted up to a factor of 60C;

One mother tincture of Agnus castus, optionally diluted up to a factor of 60C;

One mother tincture of Lobelia inflata, optionally diluted up to a factor of 60C.

According to a particular aspect of the invention, the homoeopathic composition is formulated from 6 homoeopathic strains which are diluted and added in equal masses to produce the composition. For this purpose, each of the strains can be diluted as follows:

The mother tincture of tabacum is diluted by a factor of 5D; and/or

The mother tincture of Nux Vomica is diluted by a factor of 9D; and/or

The mother tincture of Aurum metallicum is diluted by a factor of 9D; and/or

The mother tincture of Passiflora incarnata is diluted by a factor of 5D; and/or

The mother tincture of Agnus castus is diluted by a factor of 5D; and/or

The mother tincture of Lobelia inflata is diluted by a factor of 6D.

The present formulation, and in particular the homoeopathic dilution formulation, can be used to treat at least one symptom related to smoking cessation. The inventor has found that the present formulation, especially in a very diluted form, for example in the form of a homoeopathic medicine, reduces symptoms related to smoking or psychotropic drug withdrawal and can help to stop smoking.

According to a second aspect, the invention relates to a homoeopathic composition for use in the treatment of at least one symptom related to smoking cessation and/or psychotropic drug withdrawal. The invention also relates to a homoeopathic composition for accompanying treatment of a person undergoing smoking cessation and/or psychotropic drug withdrawal.

According to a third aspect, the invention relates to a method for the preparation of a homoeopathic medicinal composition. Preferably, the method comprises the following steps:

Supply of a mother tincture from each of the following 6 strains: tabacum, Nux vomica, Aurum metallicum, Passiflora incarnata, Agnus castus and Lobelia inflata;

Optionally, the dilution of at least one of the 6 mother tinctures up to a factor of 60C, the dilution of a mother tincture being carried out individually;

The mixture of the 6 mother tinctures, possibly diluted;

Possibly the addition of acceptable pharmaceutical excipients.

### DETAILED DESCRIPTION OF THE INVENTION

Within the meaning of the present invention, a homoeopathic medicine is defined according to Directive 2001/83/EC; a homoeopathic medicine thus concerns any medicine obtained from products, substances or compositions called homoeopathic strains according to a manufacturing method described by the European pharmacopoeia, or failing that, by the French pharmacopoeia.

The terms composition, formulation, medicinal composition, medicinal formulation may refer to a drug. The compositions, formulations and medicines according to the invention can be presented in a variety of different galenic forms, such as but not limited to a granule, a globule, an oral solution such as a syrup, an injectable solution, a suppository, a collyrium, a powder to be consumed orally or for cutaneous application, a cream, an ointment, a tablet, a pill, a capsule, a gum, a lozenge, a drinkable emulsion. The preparations may also be administered by means of a medical device, such as a transdermal system such as a patch or patch, an inhalation device such as a powder or pressurized inhaler, or a nebulizer.

Homoeopathic strains can be of plant, animal, chemical, mineral or organic origin.

Homoeopathic medicines are obtained by the method known as successive deconcentrations of homoeopathic strains or homoeopathic mother tinctures. This deconcentration is accomplished by successive operations of dividing the strain or mother tincture in a vehicle (dilution or trituration) to the nearest 1/100 (centesimal; 1 part strain or mother tincture and 99 parts vehicle, commonly designated “C” or “CH”, the term CH referring to “Hahnemannian centesimal”) or 1/10 (decimal; 1 part strain or mother tincture and 9 parts vehicle, commonly designated “D”, “DH” or “X”, the term DH referring to “Hahnemannian decimal”), and by successive dynamization operations (dilution followed by succussion). The number of operations thus performed defines the dilution height (or degree of dilution). Thus, and by way of example, D3 (or 3DH or 3X) means 3 successive decimal deconcentrations of the strain or mother tincture; and C3 (or 3CH or 3C) means 3 successive centesimal deconcentrations of the strain or mother tincture. The term dilution used in the present description may thus also be understood to mean that a dynamization is performed, or that the dilution is performed by trituration.

Unless otherwise specified, the term party refers to the parties en masse.

According to a first aspect, the invention relates to a homoeopathic formulation, composition or medicine comprising 6 homoeopathic mother tinctures, optionally diluted. For this purpose, there is provided a homoeopathic formulation, composition or medicine comprising, by equal mass:

One mother tincture of tabacum, optionally diluted up to a factor of 60C;

One mother tincture of Nux vomica, optionally diluted up to a factor of 60C;

One mother tincture of Aurum metallicum, optionally diluted up to a factor of 60C;

One mother tincture of Passiflora incarnata, optionally diluted to a factor of 60C;

One mother tincture of Agnus castus, optionally diluted to a factor of 60C;

One mother tincture of Lobelia inflata, optionally diluted to a factor of 60C.

A mother tincture is prepared from a strain. A mother tincture can be prepared by methods known to the person skilled in the art. The final extraction ratio of the mother tincture is 1:10, i.e. 10 grams of mother tincture are prepared from 1 gram of the drug that serves as the strain (e.g. a dry plant powder when the strain is a plant). As an example, a method of making any mother tincture may comprise the following steps: a maceration step followed by an expression step.

A macerating process may include a step of mixing the raw materials within a macerating vessel. The raw materials are usually purified water, ethanol, such as 96% ethanol, and the split drug, which is the strain from which the mother tincture will be derived. Each of the raw materials is weighed according to the manufacturing protocol of the mother tincture. Protocols for making mother tincture can be found in the reference books of the European or French Pharmacopoeia. The French Pharmacopoeia, for example, contains a homoeopathic section, which indicates the characteristics of mother tinctures (weight of dry residues, chromatography). After mixing the raw materials, the container can be closed and the mixture can macerate for a period of between 10 days and 8 weeks. During this period, the mixture can be shaken regularly, but usually at least 3 times during the maceration period.

Following the maceration stage, an expression stage is implemented. For this purpose, a cloth can be placed on a press plate, and the macerated preparation can be poured onto this cloth. The preparation is then pressed, preferably in stages, until the macerated preparation has flowed through the cloth. The liquid thus collected is the expressed liquid, and can be left to rest for a period of 48 hours.

A formulation according to the invention thus corresponds to the mixture of 6 different homoeopathic strains, the dilution height of each of which is comprised between the initial dilution of the strain within the mother tincture up to a maximum dilution corresponding to 60 CH, preferably 30 CH, and more preferably 60 DH. Each strain can be incorporated into the formulation at equivalent mass of the other strains. Thus, the formulation may comprise 1 mass or the same mass, which may be arbitrarily determined, of each of the 6 homoeopathic strains, diluted or not.

The first homoeopathic strain present in a formulation according to the invention is the homoeopathic strain tabacum, also known as Nicotiana tabacum.

The drug tabacum is made of fresh leaves of Nicotiana tabacum, harvested at the end of flowering. The mother tincture tabacum (or the strain of tabacum) corresponds to the monograph TABACUM for homoeopathic preparations of the French pharmacopoeia, 10th edition (January 1989-January 1992). It is prepared at 1/10 ethanol content of 45% V/V from freshly harvested Nicotiana tabacum leaves.

The formulation according to the invention may thus comprise 1 mass of the homoeopathic strain tabacum, a non-deconcentrated mother tincture at a dilution, or deconcentration, of at most 60 CH. Preferably, 1 mass of the strain tabacum is present at a dilution height of between 0 (i.e. mother tincture) to a dilution height of at most 60 DH. More preferably, 1 mass of the tabacum strain is present within the formulation at a dilution height between 0 (i.e. mother tincture) to a dilution height of at most 10 DH. Even more preferably, 1 mass of the tabacum strain is present in the formulation at a dilution level of 5 DH. The dilution height refers to the number of dilutions of the tabacum strain (i.e. the mother tincture).

In order to carry out the deconcentration of the tabacum strain, the Hahnemannian method can be used with ethanol as the deconcentration vehicle, preferably at 30% V/V. Such an ethanol can for example be obtained by mixing purified water with 96% ethanol corresponding to the monographs purified water (no 0008) and 96% ethanol (no 1317) of the European Pharmacopoeia respectively.

The presence of tabacum in the formulation allows it to exert a physiological action on headaches, dizziness, paleness of the face, cold sweats, obscured sensitivity with awareness of anxiety, nausea and vomiting.

The second homoeopathic strain present in the formulation according to the invention is the homoeopathic strain Nux vomica.

The drug Nux vomica is prepared by maceration of the dried seed of Strychnos Nux vomica. The Nux vomica homoeopathic strain can be constituted by the Nux vomica mother tincture corresponding to the monograph Nux Vomica for homoeopathic preparations of the French Pharmacopoeia Xth edition (2002). It is prepared at 1/10 ethanol content 65% V/V, from the dried seed of Strychnos Nux vomica, according to the monograph Nux Vomica for homoeopathic preparations of the French Pharmacopoeia 10th edition (January 1989—January 1992).

The formulation according to the invention may thus comprise 1 mass of the homoeopathic strain Nux vomica, of a non-deconcentrated mother tincture at a dilution, or deconcentration, of at most equivalent to 60 CH. Preferably, 1 mass of the Nux vomica strain is present at a dilution height between 0 (i.e. mother tincture) to a dilution height of at most 60 DH. More preferably, 1 mass of the Nux vomica strain is present within the formulation at a dilution height between 0 (i.e. mother tincture) to a dilution height of at most 10 DH. Even more preferably, 1 mass of the Nux vomica strain is present in the formulation at a dilution level of 9 DH. The dilution height refers to the number of dilutions of the Nux vomica strain (i.e. the mother tincture).

In order to deconcentrate the Nux vomica strain, the Hahnemannian method can be used with 60% V/V ethanol for the first dilution, 50% V/V ethanol for the second dilution, and 30% V/V ethanol for all subsequent dilutions. Such an ethanol can for example be obtained by mixing purified water with 96% ethanol corresponding to the monographs purified water (no 0008) and 96% ethanol (no 1317) of the European Pharmacopoeia 4th edition, respectively.

The presence of Nux vomica in the formulation allows it to exert a physiological action on the cerebrospinal system and on the gastrohepato-intestinal system, resulting in a general hyper-reflexivity, i.e. motor, psychic, visceral sensory, in association with hyperesthesia to pain

The third homoeopathic strain present in the formulation according to the invention is the homoeopathic strain Aurum metallicum.

The Aurum metallicum strain complies with the monograph Aurum metallicum for homoeopathic preparations of the French Pharmacopoeia, 10th edition (January 1993). The strain is gold (Au). The strain contains a minimum of 98% gold and a maximum of 101% gold equivalent.

The formulation according to the invention may thus comprise 1 mass of the homoeopathic strain Aurum metallicum, of a non-deconcentrated mother tincture at a dilution, or deconcentration, of at most equivalent to 60 CH. Preferably, 1 mass of the Aurum metallicum strain is present at a dilution height between 0 (i.e. mother tincture) to a dilution height of at most 60 DH. More preferably, 1 mass of the Aurum metallicum strain is present in the formulation at a dilution level between 0 (i.e. mother tincture) to a dilution level of at most 10 DH. Even more preferably, 1 mass of the Aurum metallicum strain is present in the formulation at a dilution level of 9 DH. The dilution height refers to the number of dilutions of the Aurum metallicum strain (i.e. the mother tincture).

In order to deconcentrate the strain, the vehicle is lactose when the deconcentrations are intended for oral powders.

When the deconcentrations are intended for solutions, the deconcentrations of Aurum metallicum strain are as follows:

Decimal deconcentrations: dissolve trituration D6 in purified water to obtain decimal height D7; subsequent deconcentrations are carried out using 30% V/V ethanol, prepared from purified water and 96% V/V ethanol.

Centesimal deconcentrations: dissolution of the third centesimal trituration 3C, in equal volumes of water and 60% V/V alcohol, added successively; the following deconcentrations are carried out using 30% V/V ethanol, prepared from purified water and 96% V/V ethanol.

Lactose, purified water and 96% ethanol meet the specifications of the lactose monohydrate, purified water and 96% ethanol monographs of the European Pharmacopoeia 3rd edition, respectively.

The presence of Aurum metallicum in the formulation allows it to exert a physiological action on depression, cardiovascular erethism, for example with cephalic congestion, hypertensive tendency and cardiomegaly.

The fourth homoeopathic strain present in the formulation according to the invention is the homoeopathic strain Passiflora incarnata.

The Passiflora incarnata homoeopathic strain is constituted by the Passiflora incarnata mother tincture corresponding to the monograph Passiflora incarnata for homoeopathic preparations of the French Pharmacopoeia 10th edition (1989). It is prepared at 1/10 ethanol content 65% V/V from the aerial part of Passiflora incarnata.

The formulation according to the invention may thus comprise 1 mass of the homoeopathic strain Passiflora incarnata, of a non-deconcentrated mother tincture at a dilution, or deconcentration, of at most equivalent to 60 CH. Preferably, 1 mass of the Passiflora incarnata strain is present at a dilution height between 0 (i.e. mother tincture) to a dilution height of at most 60 DH. More preferably, 1 mass of the Passiflora incarnata strain is present within the formulation at a dilution height between 0 (i.e. mother tincture) to a dilution height of at most 10 DH. Even more preferably, 1 mass of the Passiflora incarnata strain is present in the formulation at a dilution level of 5 DH. The dilution height refers to the number of dilutions of the Passiflora incarnata strain (i.e. the mother tincture).

In order to deconcentrate the Passiflora incarnata strain, the Hahnemannian method can be used with 60% V/V ethanol for the first dilution, 50% V/V ethanol for the second dilution, and 30% V/V ethanol for all subsequent dilutions. Such an ethanol can for example be obtained by mixing purified water with 96% ethanol corresponding to the monographs for purified water (no 0008) and 96% ethanol (no 1317) of the European Pharmacopoeia 4th edition, respectively.

The presence of Passiflora incarnata in the formulation according to the invention enables it to exert a physiological action on the cerebrospinal nervous system and to treat insomnia problems.

The fifth homoeopathic strain present in the formulation according to the invention is the homoeopathic strain Agnus castus.

The Agnus castus homoeopathic strain consists of the Agnus castus mother tincture, which complies with the Agnus castus monograph for homoeopathic preparations (1998) of the French Pharmacopoeia 10th edition. It is prepared at 1/10 ethanol content 65 V/V from the dried fruit of Vitex angus-castus.

The formulation according to the invention may thus comprise 1 mass of the homoeopathic strain Agnus castus, of a non-deconcentrated mother tincture at a dilution, or deconcentration, of at most equivalent to 60 CH. Preferably, 1 mass of the Agnus castus strain is present at a dilution height between 0 (i.e. mother tincture) to a dilution height of at most 60 DH. More preferably, 1 mass of the Agnus castus strain is present in the formulation at a dilution level between 0 (i.e. the mother tincture) to a dilution level of at most 10 DH. Even more preferably, 1 mass of the Agnus castus strain is present in the formulation at a dilution level of 5 DH. The dilution height refers to the number of dilutions of the Agnus castus strain (i.e. the mother tincture).

In order to deconcentrate the Agnus castus strain, the Hahnemannian method can be used with 60% V/V ethanol for the first dilution, 50% V/V ethanol for the second dilution, and 30% V/V ethanol for all subsequent dilutions. Such an ethanol can for example be obtained by mixing purified water with 96% ethanol corresponding to the monographs for purified water (no 0008) and 96% ethanol (no 1317) of the European Pharmacopoeia 4th edition, respectively.

The presence of the Agnus castus strain in the formulation allows it to exert a physiological action on sexual neurasthenia, nervous headaches, and the prevention of tachycardia.

The sixth homoeopathic strain present in the formulation according to the invention is the homoeopathic strain Lobelia inflata.

The homoeopathic strain Lobelia inflata complies with the monograph Swollen lobelia for homoeopathic preparations of the French Pharmacopoeia as to its method of manufacture and specifications. It can also be manufactured, by a maceration method, at 1/10 ethanol content of 65% V/V from the freshly flowered aerial part of Lobelia inflata, according to process 4c of the European Pharmacopoeia (no 2371). It contains a minimum of 0.01% m/m and a maximum of 0.05% m/m of total alkaloids expressed as lobeline (C22H27NO2; Mr 337.5) (adjusted content).

The formulation according to the invention may thus comprise 1 mass of the homoeopathic strain Lobelia inflata, of a non-deconcentrated mother tincture at a dilution, or deconcentration, of at most equivalent to 60 CH. Preferably, 1 mass of Lobelia inflata is present at a dilution height of between 0 (i.e. mother tincture) to a dilution height of at most 60 DH. More preferably, 1 mass of the strain Lobelia inflata is present within the formulation at a dilution height between 0 (i.e. mother tincture) to a dilution height of at most 10 DH. Even more preferably, 1 mass of the strain Lobelia inflata is present in the formulation at a dilution level of 6 DH. The dilution height refers to the number of dilutions of the strain Lobelia inflata (i.e. the mother tincture).

In order to carry out the deconcentration of the strain Lobelia inflata, the Hahnemannian method can be used with ethanol as deconcentration vehicle, preferably at 30% V/V. Such an ethanol can, for example, be obtained by mixing purified water with 96% ethanol corresponding to the monographs purified water (no 0008) and 96% ethanol (no 1317) of the European Pharmacopoeia respectively.

The presence of the Lobelia inflata strain in the formulation allows it to exert a physiological action on the symptoms of smoking cessation, in particular by reducing violent nausea and asthmatic dyspnoea with vagotonic syndrome.

In a preferred embodiment of the invention, the homoeopathic formulation comprises, by equal mass:

One mother tincture of tabacum, optionally diluted up to a factor of 60D;

One mother tincture of Nux vomica, optionally diluted up to a factor of 60D;

One mother tincture of Aurum metallicum, optionally diluted up to a factor of 60D;

One mother tincture of Passiflora incarnata, optionally diluted up to a factor of 60D;

One Agnus castus mother tincture, optionally diluted up to a factor of 60D;
One mother tincture of Lobelia inflata, optionally diluted up to a factor of 60D.

In a more preferred embodiment of the invention, the homoeopathic formulation comprises, by equal mass:

One mother tincture of tabacum, optionally diluted up to a factor of 10D;

One mother tincture of Nux vomica, optionally diluted up to a factor of 10D;

One mother tincture of Aurum metallicum, optionally diluted up to a factor of 10D;

One mother tincture of Passiflora incarnata, optionally diluted up to a factor of 10D;

One Agnus castus mother tincture, optionally diluted up to a factor of 10D;

One mother tincture of Lobelia inflata, optionally diluted up to a factor of 10D.

In a more preferred embodiment of the invention, the homoeopathic formulation comprises, by equal mass:

A mother tincture of tabacum, diluted by a factor of 5D; and/or

A mother tincture of Nux vomica, diluted by a factor of 9D; and/or

A mother tincture of Aurum metallicum, diluted by a factor of 9D; and/or

One Passiflora incarnata mother tincture, diluted by a factor of 5D; and/or

A mother tincture of Agnus castus, diluted by a factor of 5D; and/or

A mother tincture of Lobelia inflata, diluted by a factor of 6D.

In a more preferred embodiment of the invention, the homoeopathic formulation comprises, by equal mass:

A mother tincture of tabacum, diluted by a factor of 5D; and

A mother tincture of Nux vomica, diluted by a factor of 9D; and

One mother tincture of Aurum metallicum, diluted by a factor of 9D; and

One Passiflora incarnata mother tincture, diluted by a factor of 5D; and

One Agnus castus mother tincture, diluted by a factor of 5D; and

One mother tincture of Lobelia inflata, diluted by a factor of 6D.

The compositions, formulations and medicines according to the invention can be defined as pharmaceutical compositions, pharmaceutical formulations or medicines, suitable for administration to a person or patient, such as a human being. In general, these compositions, formulations or drugs are sterile, and do not comprise any compound likely to cause an undesirable response in the subject to which the invention is administered. A composition according to the invention may thus further comprise pharmaceutical excipients, such as water and/or sodium chloride, diluents, adjuvants, and/or pharmaceutical salts.

The formulation according to the invention is particularly suitable for the treatment of symptoms related to smoking cessation and/or psychotropic drug withdrawal.

The formulation according to the invention is particularly suitable for the accompanying treatment of people stopping their smoking and/or psychotropic drug consumption.

The formulation according to the invention is particularly suitable as an accompanying treatment for smoking cessation therapy, in addition to another treatment such as another drug treatment or consumption of nicotine derivatives.

The invention also relates to a method for the preparation of a homoeopathic medicinal composition. The method comprises the following steps:

Supply of a mother tincture from each of the following 6 strains: tabacum, Nux vomica, Aurum metallicum, Passiflora incarnata, Agnus castus and Lobelia inflata;
Optionally, the dilution of at least one of the 6 mother tinctures up to a factor of 60C, the dilution of each mother tincture being carried out individually;

The mixing with equivalent masses of the 6 mother tinctures, possibly diluted;

Possibly the addition of acceptable pharmaceutical excipients.

Optionally, the dilution of at least one of the 6 mother tinctures may be carried out up to a factor of 60D, the dilution of each mother tincture being carried out individually.

Preferably, each mother tincture is diluted up to a factor of 60C, more preferably up to a factor of 60D.

Preferably, the mother tincture of tabacum is diluted by a factor of 5D; the mother tincture of Nux vomica is diluted by a factor of 9D; the mother tincture of Aurum metallicum is diluted by a factor of 9D; the mother tincture of Passiflora incarnata is diluted by a factor of 5D; the mother tincture of Agnus castus is diluted by a factor of 5D; and the mother tincture of Lobelia inflata is diluted by a factor of 6D.

In a preferred embodiment, the method further comprises a step of final dilution of the formulation with a sodium chloride solution, in order to obtain a final 0.9% isotonic solution.

In a particular embodiment, a sterilization step of the formulation may be implemented.

In a particular embodiment, the composition according to the invention is in a form acceptable for injection, more particularly in a form acceptable for transdermal or subcutaneous injection. The composition can thus be administered, for example by means of a syringe to a patient.

In a particular embodiment of the invention, the composition is administered only once to a patient. This single administration may comprise taking, in particular orally, or injecting, a sample of the composition or a plurality of samples of the composition, in particular depending on the patient's presumed dependence on nicotine or the psychotropic drug whose effects the treatment seeks to annihilate or reduce during withdrawal. In another embodiment, especially when patients maintain a troublesome dependence during treatment or when the dependence is estimated to be significant, the composition may be administered several times, over a period of time that may vary according to various clinical elements, such as the patient's presumed dependence, the effects of withdrawal, and the desire to use tobacco or a psychotropic drug again. Assumed dependence can be assessed by known methods; for example, the Fagerstrom test can be used to determine the level of dependence of a patient.

### BRIEF DESCRIPTION OF THE FIGURES

FIG. 1 shows the socio-professional data associated with the patient cohort. FIG. 1A shows the age distribution of the patients in the study, and FIG. 1B shows the distribution of the socio-professional categories of the patients within the cohort.

FIG. 2 illustrates the success rate by gender of smoking cessation in patients who received a composition according to the invention. By success, it should be understood that the patients reported abstinence from tobacco consumption after administration of the composition according to the invention.

FIG. 3 is a graph showing the proportion of patients who stopped smoking and those who used tobacco after administration of the formulation, as a function of their stated motivation to stop smoking.

FIG. 4 is a graph showing the relationship between the duration of abstinence reported by patients after being treated with a composition according to the invention and the duration of this abstinence.

FIG. 5 is a graph illustrating the abstinence rate of patients according to their estimated dependence according to the Fagerstrom test.

FIG. 6 is a graphical representation of the time frame in which the effects of the compound on smoking cessation are experienced by patients. The effects are, for example, the phenomenon known as “craving”, sleep or eating disorders, and emotional instability.

### EXPERIMENTAL RESULTS

Within psychiatric clinics specialising in particular in the analysis, monitoring and treatment of addictions, a certain number of patients have been monitored in the context of smoking cessation support with the administration of a formulation according to the invention. Under the coordination of psychiatrists, tobaccologists and addictologists, an independent, multicentre, real-life clinical study is currently being carried out to assess the impact of the formulation on smoking abstinence; this study provides for the follow-up of patients over a period of 24 months; the results presented here cover a period of approximately 18 months. A total of seven physicians were involved in the implementation and monitoring of the treatment.

The participants were given, at one time, two subcutaneous injections of 1 ml each of the homoeopathic formulation comprising the following compounds in equal masses:
A mother tincture of tabacum, diluted by a factor of 5D; and

A mother tincture of Nux vomica, diluted by a factor of 9D; and

One mother tincture of Aurum metallicum, diluted by a factor of 9D; and

One Passiflora incarnata mother tincture, diluted by a factor of 5D; and

One Agnus castus mother tincture, diluted by a factor of 5D; and

One mother tincture of Lobelia inflata, diluted by a factor of 6D.

Over the course of the study (approximately 18 months), of the patients treated with a formulation according to the invention, 778 subjects gave their agreement in principle to participate in this study. At the time of the post-treatment contact, 554 of them responded, thus constituting the cohort for the study of the effects of the formulation on smoking cessation.

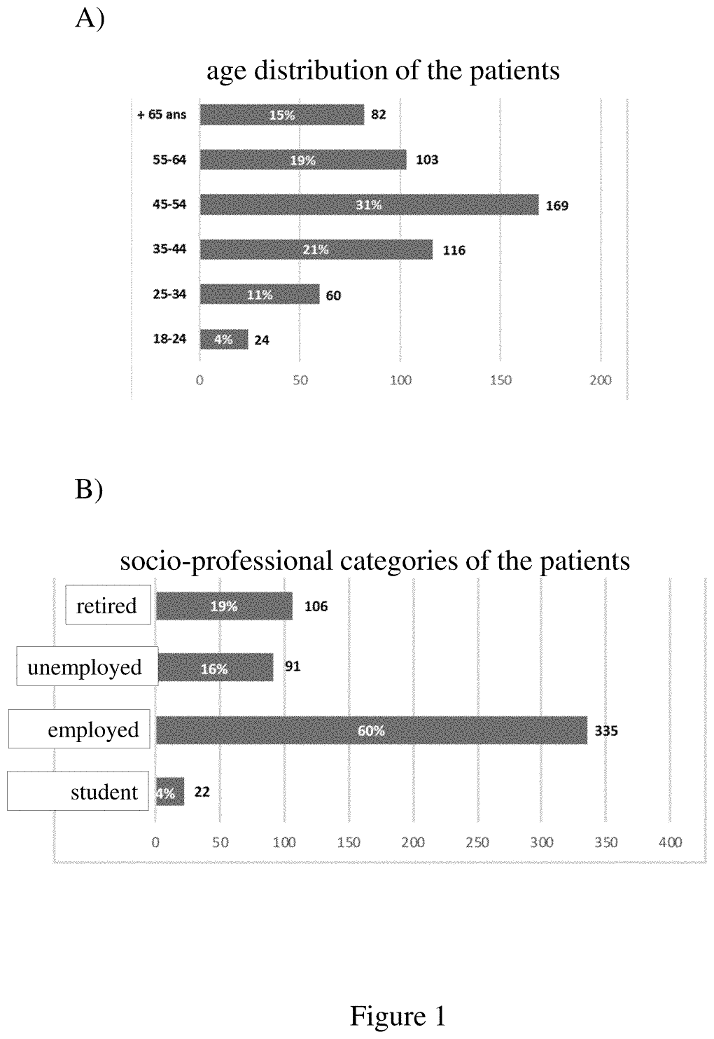

The cohort is 45% female (n=248), and therefore 55% male (n=306). The age and socio-professional category of the subjects were noted. The graphs in FIG. 1A and FIG. 1B illustrate these two data items for the entire cohort.

### Results:

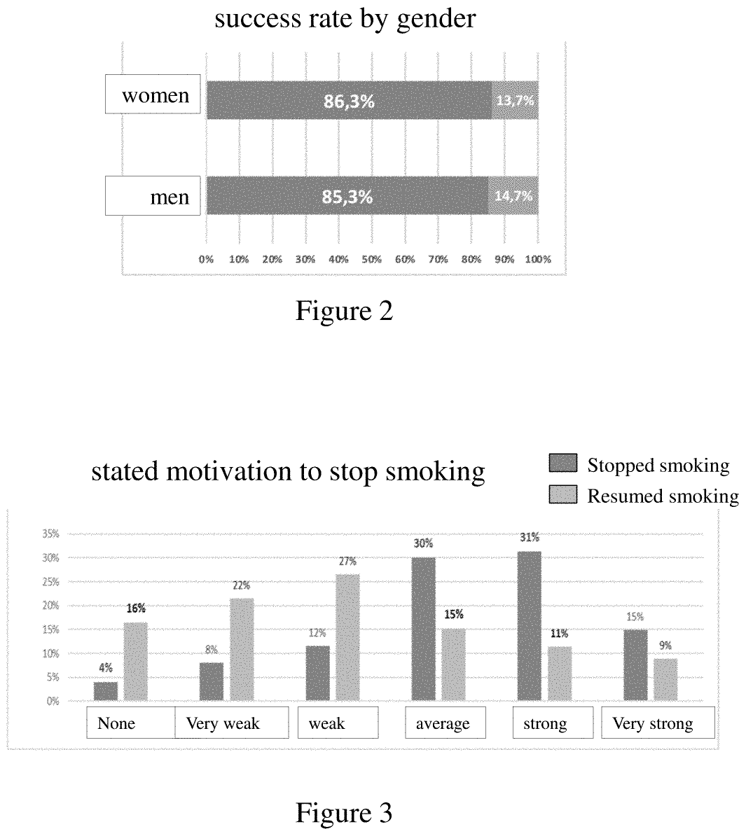

Of the 554 subjects in the study, the reported abstinence from smoking after receiving a subcutaneous injection of the homoeopathic formulation was 85.7% (see graph in FIG. 2). This rate of declared abstinence seems to be independent of the patient's age and socio-professional category. The gender of the patient also seems to have no impact on withdrawal, as women and men report abstinence in similar proportions (86.3% and 85.3% respectively; see FIG. 2). In other words, only about 15% of patients report a recurrence of their smoking within the first few months of treatment.

The study also shows that cessation is particularly satisfactory for people who reported having a moderate to strong motivation to quit smoking, since people who reported being moderately motivated to quit do not resume smoking in satisfactory proportions (see graph in FIG. 3). The satisfaction score for the medical consultation is very high among patients who have quit smoking, exceeding 8.2/10, supporting the importance that patients place on treatment in helping them quit smoking.

It is also important to note that no patient has reported any side effects following the injection of the formulation. On the contrary, some treatments known in the prior art for smoking cessation are accompanied by mild to severe side effects.

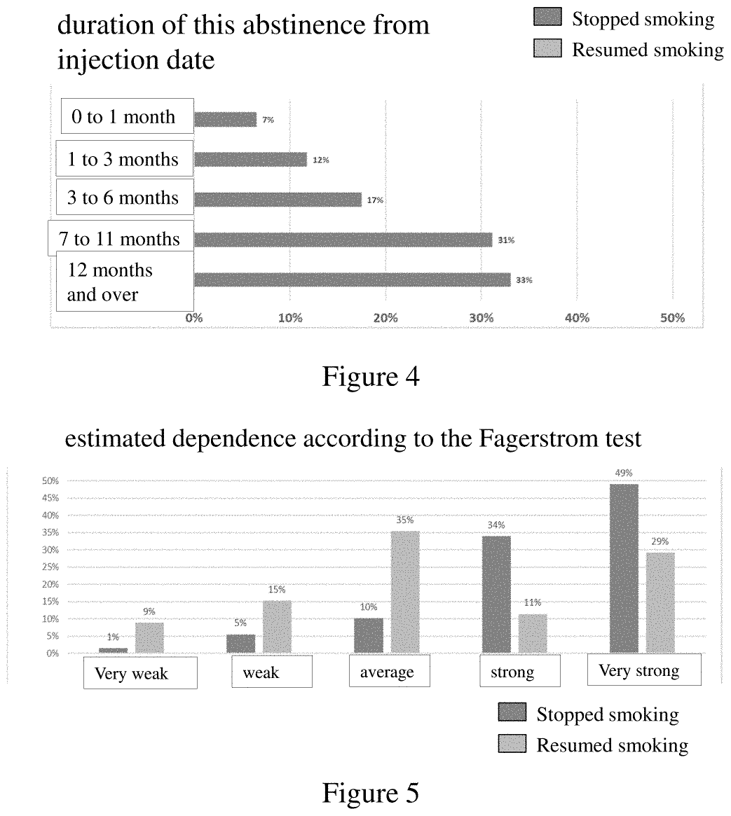

The number of patients who have stopped smoking is thus higher than 85%, much higher than the usual success rates of prior art cessation strategies, but in addition, the long-term result is also satisfactory, since 66% of the patients declared that they had not used tobacco 6 months after receiving the treatment and, for more than 33% of them, after one year. These data items are illustrated in FIG. 4. These withdrawal rates are higher than the rates usually observed in therapies known in the prior art, with a very high tolerance threshold. This recurrence rate is lower than the results generally observed in patients using other means of withdrawal (other formulations of traditional medicine, homoeopathic medicine or via alternative medicines such as hypnosis or acupuncture). For example, treatment with varenicline (marketed under the brand name Champix® in Europe) results in abstinence at 12 months of 14% to 22% depending on the study. However, varenicline has serious side effects and is under increased surveillance by the French National Agency for the Safety of Medicines and Health Products, whereas the patients in this study reported no drug-related side effects.

Another point to mention concerns the results observed in patients with a high level of dependency. People with a high or very high dependence according to the Fagerstrom test carried out before treatment show a significant rate of abstinence after treatment: more than 83% of patients who stopped smoking had a high or very high dependence level, and only 40% of people with a high or very high dependence score resumed smoking after treatment (see FIG. 5).

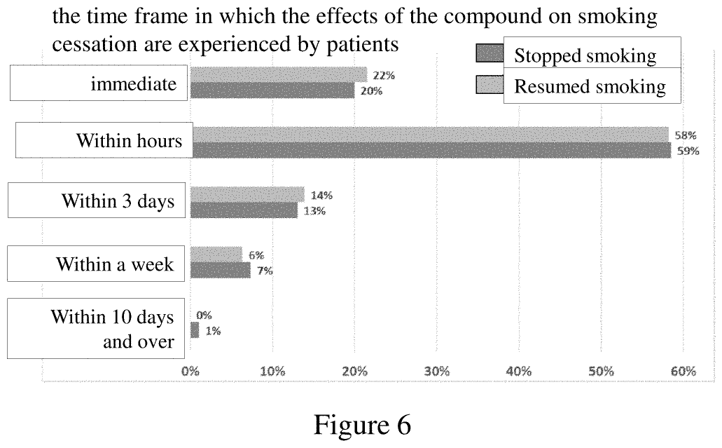

The effects of withdrawal following an injection are relatively rapid, with effects felt within hours of the treatment session in about 60% of patients, and even immediate effects in about 20% of cases (see FIG. 6).

The effects were estimated by means of individual questionnaires whose answers were collected in writing or by telephone by the team in charge of this study.

1. A homoeopathic composition comprising:
A mother tincture of tabacum, optionally diluted up to a factor of 60C;
One mother tincture of Nux vomica, optionally diluted up to a factor of 60C;
A mother tincture of Aurum metallicum;
A mother tincture of Passiflora incarnata;
A mother tincture of Agnus castus;
A mother tincture of Lobelia inflata.

2. A homoeopathic composition of claim 1, wherein the mother tincture of tabacum is diluted by a factor of 5D.

3. A homoeopathic composition of claim 1, wherein the mother tincture of Nux vomica is diluted by a factor of 9D.

4. A homoeopathic composition of claim 1, wherein the mother tincture of Aurum metallicum is diluted by a factor of 9D.

5. A homoeopathic composition of claim 1, wherein the mother tincture of Passiflora incarnata is diluted by a factor of 5D.

6. A homoeopathic composition of claim 1, wherein the Agnus castus mother tincture is diluted by a factor of 5D.

7. A homoeopathic composition of claim 1, wherein the mother tincture of Lobelia inflata is diluted by a factor of 6D.

8. A homoeopathic composition according to claim 1, comprising, by equal mass
A mother tincture of tabacum, diluted by a factor of 5D; and
A mother tincture of Nux vomica, diluted by a factor of 9D; and
One mother tincture of Aurum metallicum, diluted by a factor of 9D; and
One Passiflora incarnata mother tincture, diluted by a factor of 5D; and
One Agnus castus mother tincture, diluted by a factor of 5D; and
One mother tincture of Lobelia inflata, diluted by a factor of 6D.

9. A method for the treatment of at least one symptom related to smoking cessation and/or psychotropic drug withdrawal, and/or for a treatment accompanying a person in a smoking or psychotropic drug withdrawal situation comprising the step of:
administering a homeopathic composition of claim 1 to a patient in need.

10. The method according to claim 9, wherein the administration of the composition is made subcutaneously.

11. A method for the preparation of a homoeopathic medicinal composition, comprising the following steps:
supplying of a mother tincture from each of the following 6 strains: tabacum, Nux vomica, Aurum metallicum, Passiflora incarnata, Agnus castus and Lobelia inflata;
mixing the 6 mother tinctures.

12. A method according to claim 11, further comprising a final dilution step with a sodium chloride solution in order to finally obtain a 0.9% isotonic solution, followed by a sterilization step of the composition.
